(19) United States
(12) Patent Application Publication
(10) Pub. No.: US 2002/0010216 A1
(43) Pub. Date: Jan. 24, 2002

(54) New drug combinations

(76) Inventors: Karen Rogosky, Robbinsville, NJ (US); Deborah Jorn, Warren, NJ (US)

Correspondence Address: Andrew M. Solomon, Pharmacia & UpJohn Company, Global Intellectual Property, 301 Henrietta Street, Kalamazoo, MI, 49001, US

(21) Appl. No.: 09/792,718

(22) Filed: Feb. 23, 2001

Related U.S. Application Data
(60) Provisional application No. 60/184,790, filed on Feb. 24, 2000.

(51) Int. Cl. A61K 31/135

(52) U.S. Cl. 514/649; 514/651

(57) ABSTRACT

A composition comprising:

(a) a pharmaceutically effective amount of one or more norepinephrine reuptake inhibitors or a pharmaceutically effective salt thereof, and

(b) a pharmaceutically effective amount of one or more antimuscarinic agents or a pharmaceutically effective salt thereof is provided. The composition is useful in treating disorders or diseases of the central nervous system, and particularly useful in treating incontinence.

### CROSS-REFERENCE TO RELATED APPLICATIONS

This application claims the benefit of the following provisional application: U.S. Ser. No. 60/184790, filed Feb. 24, 2000.

### BACKGROUND OF THE INVENTION

1. Field of the Invention

This invention describes new treatments that should provide for a fast acting rapid onset of relief from several nervous system disorders, and it involves the administration of a norepinephrine reuptake inhibitor, preferably a selective norepinephrine reuptake inhibitor, most preferably the drug reboxetine, in combination with an antimuscarinic agent, preferably tolterodine. In particular, the combination is to be used to treat incontinence.

2. Technology Description

The introduction of tricyclic antidepressants in the early 1960s has provided a major advance in the treatment of neuropsychiatric disorders. Reactive and endogenous depressions, diagnoses formerly carrying grave prognostic implications, have become, with the introduction of the tricyclics, manageable disorders with a much smaller toll on the patient and the society as a whole.

The early tricyclic compounds were reuptake inhibitors of all the catecholamines released in the synaptic cleft, thus resulting in prolongation and enhancement of the dopamine (DA), noradrenaline (NA) and serotonin (5-hydroxytryptamine=5-HT) action. Lack of selectivity also causes undesired side effects particularly on the acetylcholine (especially the muscarinic component), and histamine mediated neurotransmission.

Because of these unwanted pharmacodynamic activities, cognitive impairment, sedation, urinary and gastrointestinal tract disturbances, and increased intraocular pressure were limiting factors in the clinical use of these compounds and often required discontinuation of treatment. Of utmost concern were also the cardiac toxic effects and the proconvulsant activity of this group of drugs.

More recently, selective reuptake inhibitors for serotonin (SSRI) have been introduced with definite advantages in regard to fewer side effects without loss of efficacy. Fluoxetine is an example of such an inhibitor that has had a great amount of commercial success.

Another class of compounds that has been proposed for use in the treatment of depression is selective norepinephrine reuptake inhibitors. Lower-than-normal levels of norepinephrine are associated with a variety of symptoms including lack of energy, motivation, and interest in life. Thus, a normal level of norepinephrine is essential to maintaining drive and capacity for reward. These neurotransmitters travel from the terminal of a neuron across a small gap (i.e., the synaptic cleft) and bind to receptor molecules on the surface of a second neuron. This binding elicits intracellular changes that initiate or activate a response or change in the postsynaptic neuron. Inactivation occurs primarily by transport (i.e., reuptake) of the neurotransmitter back into the presynaptic neuron. Abnormality in noradrenergic transmission results in various types of depression, mental, behavioral, and neurological disorders attributed to a variety of symptoms including a lack of energy, motivation, and interest in life. See generally, R. J. Baldessarini, “Drugs and the Treatment of Psychiatric Disorders: Depression and Mania” in Goodman and Gilman's The Pharmacological Basis of Therapeutics, McGraw-Hill, NY, N.Y., pp. 432-439 (1996).

Examples of norepinephrine reuptake inhibitors (both selective and not selective) include, but are not limited to the following: tandamine (CAS 42408-80-0; U.S. Pat. No. 3,904,617; U.S. Pat. No. 4,118,394), pirandamine (CAS 42408-79-7; U.S. Pat. No. 3,995,052), ciclazindol (CAS 37751-39-6; U.S. Pat. No. 3,891,644; U.S. Pat. No. 3,957,819; U.S. Pat. No. 3,976,645), fluparoxan (U.S. Pat. No. 4,880,801), lortalamine (CAS 70384-91-7; U.S. Pat. No. 4,201,783), talsupram (CAS 21489-20-3), talopram (CAS 7182-51-6), prindamine, nomifensine (U.S. Pat. No. 3,577,424), viloxazine (U.S. Pat. No. 3,712,890), tomoxetine (U.S. Pat. No. 4,314,081), duloxetine (U.S. Pat. No. 5,023,269), venlafaxine (U.S. Pat. No. 4,535,186), milnacipran (U.S. Pat. No. 4,478,836) and reboxetine (U.S. Pat. No. 4,229,449).

Tomoxetine, (R)-(−)-N-methyl-3-(2-methylphenyoxy)-3-phenylpropylamine is disclosed in U.S. Pat. No. 4,314,081. Reboxetine, 2-[alpha-(2-ethoxy)phenoxybenzyl]morpholine is disclosed in U.S. Pat. No. 4,229,449. Reboxetine includes both the racemate, as well as the (−)(R,R) and (+)(S,S) enantiomers. This product is also identified by the following trademarks: VESTRA, PROLIFT, NOREBOX and ERDONAX. Duloxetine, N-methyl-3-(1-naphthalenyloxy)-3-(2-thienyl) propanamine is disclosed in U.S. Pat. No. 4,956,388. It is usually administered as the hydrochloride salt and as the (+) enantiomer. Venlafaxine is identified as Compound A of U.S. Pat. No. 4,761,501. Milnacipran, N,N-diethyl-2-aminomethyl-1-phenylcyclopropanecarboxamide is disclosed in U.S. Pat. No. 4,478,836. To the extent necessary for completion, these above-cited documents are expressly incorporated by reference.

Antimuscarinic agents can be used to treat urinary incontinence. Examples of antimuscarinic agents include, but are not limited to the following: tolterodine, propiverine, oxybutynin, trospium, darifenacin, temiverine, ipratropium.

Tolterodine, Phenol, 2-[3-[bis(1-methylethyl)amino]-1-phenylpropyl]-4-methyl-, (R)-, an antimuscarinic with a high degree of bladder selectivity, has been developed and launched by Pharmacia & Upjohn under the DETROL® trademark for the treatment of incontinence associated with an overactive bladder. This product is disclosed in U.S. Pat. No. 5,382,600. The active metabolites of tolterodine are disclosed in U.S. Pat. No. 5,559,269, with a preferred metabolite being 2-[(1R)-3-(diisopropylamino)-1-phenylpropyl]-4-(hydroxymethyl) phenol in either its native or hydroxy(phenyl) acetate salt form.

Propiverine is 1-methyl-4-piperidyl alpha.,.alpha.-diphenyl-.alpha.-(n-propoxy)acetate and is disclosed in East German Patent No. 106643 and in CAS 82-155841s (1975). Oxybutynin is 4-(Diethylamino)-2-butynylalpha-phenylcyclohexaneglycolate and is disclosed in UK Patent No. 940540. Trospium is 3alpha-Hydroxyspiro[1alphaH,5alphaH-nortropane-8,1′-pyrrolidinium]chloride benzilate and is disclosed in U.S. Pat. No. 3,480,623. Darifenacin is 3-Pyrrolidineacetamide, 1-[2-(2,3-dihydro-5-benzofuranyl)ethyl]-alpha,alpha-diphenyl-, and is disclosed in U.S. Pat. No. 5,096,890. Temiverine is Benzeneacetic acid, .alpha.-cyclohexyl-.alpha.-hydroxy-, 4-(diethylamino)-1,1-dimethyl-2-butynyl ester and is disclosed in U.S. Pat. No. 5,036,098 and ipratropium is 8-isopropylnoratropine methobromide and disclosed in U.S. Pat. No. 3,505,337.

Despite the above advances in the art, it would be desirable to develop a pharmaceutical composition that would have both the benefits of the norepinephrine reuptake inhibitors and that of the antimuscarinic agents.

### BRIEF SUMMARY OF THE INVENTION

In accordance with the present invention a novel pharmaceutical composition is provided. More specifically, the composition combines one or more selective norepinephrine reuptake inhibitors with one or more antimuscarinic agents, preferably tolterodine. The composition is considered to be particularly effective against incontinence in general, and more particularly stress incontinence.

A first embodiment of the present invention provides a composition comprising:

(a) a pharmaceutically effective amount of one or more norepinephrine reuptake inhibitors or a pharmaceutically effective salt thereof; and

(b) a pharmaceutically effective amount of one or more antimuscarinic agents or a pharmaceutically effective salt thereof.

In particularly preferred embodiments, component (a) comprises reboxetine in either its enantiomeric or racemic form and component (b) comprises tolterodine, including its active metabolites.

Yet another embodiment of the present invention provides a method for treating or preventing incontinence or diseases or disorders of the central nervous system comprising administering a therapeutically effective amount of the above composition to a mammal. In most instances, the mammal will be a human, and the disease or disorder to be treated is incontinence.

A further embodiment of the present invention comprises the use of the above composition to prepare a medicament for treating or preventing incontinence or diseases or disorders of the central nervous system.

An object of the present invention is to provide novel compositions having biological activity.

A further object of the present invention is to provide a method for treating or preventing incontinence or diseases of the central nervous system by using the novel compositions of the present invention.

An additional object of the present invention is to provide an effective treatment for incontinence.

These, and other objects, will readily be apparent to those skilled in the art as reference is made to the detailed description of the preferred embodiment.

### DETAILED DESCRIPTION OF THE PREFERRED EMBODIMENT

In describing the preferred embodiment, certain terminology will be utilized for the sake of clarity. Such terminology is intended to encompass the recited embodiment, as well as all technical equivalents which operate in a similar manner for a similar purpose to achieve a similar result. To the extent that any pharmaceutically active compound is disclosed or claimed, it is expressly intended to include all active metabolites produced in vivo, and, is expressly intended to include all enantiomers, isomers or tautomers where the compound is capable of being present in its entantiomeric, isomeric or tautomeric form.

The present invention provides a novel composition which is a combination of different chemical entities, more specifically, the first entity being a norepinephrine reuptake inhibitor and the second being an antimuscarinic agent.

The first component is a norepinephrine reuptake inhibitor, with selective norepinephrine reuptake inhibitors being particularly preferred. This list of compounds includes, but is not limited to the following: tandamine, pirandamine, ciclazindol, fluparoxan, lortalamine, talsupram, talopram, prindamine, nomifensine, viloxazine, tomoxetine, duloxetine, venlafaxine, milnacipran and reboxetine, with reboxetine being particularly preferred.

Examples of pharmaceutically effective salts for the selective norepinephrine reuptake inhibitor include, but are not limited to salts prepared from pharmaceutically acceptable acids or bases, including organic and inorganic acids and bases. When the preferred compound of use is basic (for example reboxetine), salts may be prepared from pharmaceutically acceptable acids. Suitable pharmaceutically acceptable acids include acetic, benzenesulfonic (besylate), benzoic, p-bromophenylsulfonic, camphorsulfonic, carbonic, citric, ethanesulfonic, fumaric, gluconic, glutamic, hydrobromic, hydrochloric, hydroiodic, isethionic, lactic, maleic, malic, mandelic, methanesulfonic (mesylate), mucic, nitric, oxalic, pamoic, pantothenic, phosphoric, succinic, sulfuric, tartaric, p-toluenesulfonic, and the like. Examples of such pharmaceutically acceptable salts include, but are not limited to, acetate, benzoate, hydroxybutyrate, bisulfate, bisulfite, bromide, butyne-1,4-dioate, carpoate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, and the like.

In particularly preferred embodiments the selective norepinephrine reuptake inhibitor is reboxetine, 2-[α-((2-ethoxyphenoxy)benzyl]-morpholine, and its pharmaceutically acceptable salts, in either its enantiomeric (particularly the (S,S) enantiomer) or racemic form. Synthesis of racemic reboxetine is described in greater detail in U.S. Pat. No. 4,229,449. Individual stereoisomers of reboxetine can be obtained by resolution of the racemic mixture of enantiomers using conventional methods generally known by those skilled in the art. Such methods include, but are not limited to, resolution by simple crystallization and chromatographic techniques, for example, as set forth in GB 2,167,407. Other methods of preparation are described in U.S. Pat. No. 5,068,433 and U.S. Pat. No. 5,391,735. Reboxetine can be a free base form, or it can be in salt form, preferably the methanesulfonate salt (also called reboxetine mesylate). To the extent necessary for completion, the above patents are expressly incorporated by reference.

The selection of the dosage of the first component is that which can provide relief to the patient. As is well known, the dosage of this component depends on several factors such as the potency of the selected specific compound, the mode of administration, the age and weight of the patient, the severity of the condition to be treated, and the like. This is considered to be within the skill of the artisan and one can review the existing literature on the components to determine optimal dosing. To the extent necessary for completion, the synthesis of the components and dosages described in the patents or CAS documents referenced in the Technology Description portion of this document are expressly incorporated by reference

Desirably, when reboxetine is selected as the active agent, the daily dose contains from about 0.1 mg. to about 10 mg. More preferably, each dose of the component contains about 0.5 to about 8 mg of the active ingredient, and even more preferably, each dose contains from about 0.5 to about 5 mg of the active ingredient. This dosage form permnits the full daily dosage to be administered in one or two oral doses. This will allow for final formulations containing 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9. 2.0, 2.1, 2.2, 2.3, 2.4, or 2.5 mg of active. More than once daily or twice daily administrations (e.g., 3, 4, 5 or 6 administrations per day) are also expressly contemplated herein.

The average daily adult dosage of the other norepinephrine reuptake inhibitors is as follows. The dosages expressly include all numerical values, whole or fractional, within the stated range. Pediatric dosages may be less.

| Component | Average Daily Dosage (mg/day/patient) |
|---|---|
| Tandamine | 7.5 to 3750 |
| Pirandamine | 7.5 to 3750 |
| Ciclazindol | 5 to 500 |
| Fluparoxan | .75 to 750 |
| Lortalamine | 1 to 200 |
| Talsupram | 1 to 3750 |
| Talopram | 1 to 3750 |
| Prindamine | 1 to 3750 |
| Nomifensine | 1 to 80 |
| Viloxazine | 1 to 3750 |
| Tomoxetine | 1 to 200 |
| Duloxetine | 5 to 500 |
| Venlafaxine | 2 to 200 |
| Milnacipran | 7.5 to 75 |

The second component comprises one or more antimuscarinic agents. Examples of such agents include tolterodine, propiverine, oxybutynin, trospium, darifenacin, temiverine and ipratropium. Particularly preferred is tolterodine.

The chemical name of tolterodine is Phenol, 2-[3-[bis(1-methylethyl)amino]-1-phenylpropyl]-4-methyl-, (R)-, including its pharmaceutically active salts, such as those described above with respect to the first active component and its active metabolites that are produced in vivo. Synthesis of tolterodine is disclosed in U.S. Pat. No. 5,382,600. The metabolites are disclosed in U.S. Pat. No. 5,559,269.

To the extent necessary for completion, these patents are expressly incorporated by reference. This compound is particularly useful as an anticholingeric agent, and more specifically for the treatment of incontinence.

As is well known, the dosage and administrative regimen (i.e., one, two, three or more administrations per day) of the second component depends on the factors referred to in connection with the dosage selection of the first component. To the extent necessary for completion, the synthesis of the components and dosages described in the patents or CAS documents referenced in the Technology Description portion of this document are expressly incorporated by reference. The average adult daily dosage of the second component is from about 0.05 mg to about 5 mg per kilogram of body weight, administered in one or more doses, e.g. containing from about 0.05 to about 250 mg each. The dosages expressly include all numerical values, whole or fractional, within the stated range. Pediatric dosages may be less.

Compositions of the present invention can conveniently be administered in a pharmaceutical composition containing the active components in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). To the extent necessary for completion, this reference is hereby incorporated by reference. The compositions of the present invention can be administered parenterally (for example, by intravenous, intraperitoneal, subcutaneous or intramuscular injection), topically, orally, intranasally, intravaginally, or rectally, with oral administration being particularly preferred.

For oral therapeutic administration, the inventive composition may be combined with one or more excipients and used in the form of ingestible tablets, buccal tablets, troches, capsules, elixirs, suspensions, syrups, wafers, chewing gums, foods and the like. Such compositions and preparations should contain at least 0.1% of active compound. The percentage of the compositions and preparations may, of course, be varied and may conveniently be between about 0.1 to about 100% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active components may be incorporated into sustained-release preparations and devices including, but not limited to, those relying on osmotic pressures to obtain a desired release profile. Once daily formulations for each of the active components are specifically included.

The inventive composition, containing the two active components, may be administered in the same physical form or concomitantly according to the above-described dosages and in the above-described delivery vehicles. The dosages for each active component can be measured separately and can be given as a single combined dose or given separately. They may be given at the same or at different times as long as both actives are in the patient at one time over a 24-hour period. Concomitant or concurrent administration means the patient takes one drug within about 5 minutes of taking the other drug. Because the goal is to provide rapid symptomatic relief to the patient, in most cases when treatment is started the two drugs would be administered to the patient close in time and typically concomitantly; thereafter, the timing of each drug's administration may not be as important.

The inventive composition is used to treat incontinence or any of the diseases or disorders of the central nervous system. Such diseases and disorders are defined in The Diagnostic and Statistical Manual of Mental Disorders-IV (DSM-IV) (American Psychiatric Association (1995)). To the extent necessary for completion, the contents of this reference and all of the defined diseases or disorders are expressly incorporated by reference. Also considered is the treatment of incontinence of any type. Representative diseases or disorders include, but are not limited to the following: obesity, depression, schizophrenia, a stress related disease (e.g. general anxiety disorder), panic disorder, a phobia, obsessive compulsive disorder, post-traumatic-stress syndrome, immune system depression, incontinence, a stress induced problem with the urinary, gastrointestinal or cardiovascular system (e.g., stress incontinence), neurodegenerative disorders, autism, chemotherapy-induced vomiting, hypertension, migraine headaches, cluster headaches, sexual dysfunction in a mammal (e.g. a human), addictive disorder and withdrawal syndrome, an adjustment disorder, an age-associated learning and mental disorder, anorexia nervosa, apathy, an attention-deficit disorder due to general medical conditions, attention-deficit hyperactivity disorder, bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, a movement disorder (e.g., Tourette's syndrome), oppositional defiant disorder, a pain disorder, peripheral neuropathy, post-traumatic stress disorder, premenstrual dysphoric disorder, a psychotic disorder, seasonal affective disorder, a sleep disorder, a specific developmental disorder, and selective serotonin reuptake inhibition (SSRI) “poop out” syndrome. Treatment of the above diseases or disorders is accomplished by delivering a therapeutically effective amount of the inventive composition to a mammal. In most cases this will be a human being, but treatment of food animals (e.g., livestock and poultry) and companion animals (e.g., dogs, cats and horses) is expressly covered herein.

In particular, the inventive composition is to be used in the treatment of incontinence (i.e., stress incontinence, genuine stress incontinence, and mixed incontinence). Stress urinary incontinence is a symptom describing involuntary loss of urine on carrying out any activity that raises intra-abdominal pressure such as coughing or sneezing. Stress incontinence is also a clinical sign, that is the observation by a care giver of a jet of urine escaping from the urethral meatus (opening) when the patient coughs or strains. Genuine Stress Incontinence (urge incontinence) is the pathological diagnosis of an incompetent urethral sphincter as diagnosed by Urodynamic testing. Mixed incontinence is stress incontinence in combination with urge incontinence. The latter is a part of the symptom complex of the Overative Bladder. Retention may be due to outflow obstruction (e.g., high urethral pressure), poor detrusor (bladder muscle) contractility or lack of coordination between detrusor contraction and urethral relaxation. The inventive drug combination can be used in connection with stress incontinence, urge incontinence or mixed incontinence.

The novel composition is expected to provide rapid relief to those suffering from the above diseases or disorders with a minimal amount of deleterious side effects.

The invention is described in greater detail by the following non-limiting example.

### EXAMPLE 1

A pharmaceutical composition is prepared by combining reboxetine in either its racemic or (S,S) entantiomeric form with tolterodine in a pharmaceutically acceptable carrier. The composition contains respective amounts of reboxetine and tolterodine to deliver on a daily basis between about 0.1 mg to about 10 mg reboxetine and between about 0.05 mg to about 4 mg of tolterodine per kilogram of patient body weight (for example, 3 mg to 240 mg tolterodine for a person weighing 60 kg). The composition is administered to a patient for the treatment of incontinence, and particularly stress incontinence, urge incontinence or mixed incontinence.

### EXAMPLE 2

A first pharmaceutical composition is prepared by combining reboxetine in either its racemic or +(S,S) enantiomeric form in a pharmaceutically acceptable carrier such that it can deliver between about 0.1 mg to about 10 mg reboxetine on a daily basis. A second pharmaceutical composition is prepared by combining tolterodine in a pharmaceutically acceptable carrier such that it can deliver between about 0.05 mg to about 4 mg of tolterodine per kilogram of patient body weight on a daily basis. The first composition is administered to a patient suffering from one or more forms of incontinence once, twice, three times, four times or six times daily such that the daily dosage is between about 0.1 to about 10 mg. The second composition is administered to the same patient at the same time as the administration of the first composition or any time within 24 hours of the administration of the first composition once, twice, three times, four times or six times daily such that the daily dosage is between about 0.05 mg to about 4 mg of tolterodine per kilogram of patient body weight. Alternatively, the second composition could first be administered, followed by the administration of the first composition as disclosed at the same time, or within 24 hours thereof.

Having described the invention in detail and by reference to the preferred embodiments thereof, it will be apparent that modifications and variations are possible without departing from the scope of the appended claims.

What is claimed is:

1. A composition comprising:
(a) a pharmaceutically effective amount of one or more norepinephrine reuptake inhibitors or a pharmaceutically effective salt thereof; and
(b) a pharmaceutically effective amount of one or more antimuscarinic agents or a pharmaceutically effective salt thereof.

2. The composition according to claim 1 wherein component (a) is selected from the group consisting of tandamine, pirandamine, ciclazindol, fluparoxan, lortalamine, talsupram, talopram, prindamine, nomifensine, viloxazine, tomoxetine, duloxetine, venlafaxine, milnacipran and reboxetine and mixtures thereof and wherein component (b) is selected from the group consisting of tolterodine, propiverine, oxybutynin, trospium, darifenacin, temiverine and ipratropium and mixtures thereof.

3. The composition according to claim 2 wherein component (a) is reboxetine in either its racemic or +(S,S) enantiomeric form and component (b) is tolterodine, its active metabolites or mixtures thereof.

4. The composition according to claim 3 containing between about 0.1 mg to about 10 mg reboxetine and between about 0.05 mg to about 4 mg of tolterodine per kilogram of patient body weight.

5. The composition according to claim 1 wherein component (a) and component (b) are maintained in the same delivery vehicle.

6. The composition according to claim 1 wherein component (a) and component (b) are maintained in different delivery vehicles.

7. A method for treating incontinence or a disease or disorder of the central nervous system in a mammal comprising administering to said mammal a pharmaceutically effective amount of a composition comprising:
(a) a pharmaceutically effective amount of one or more norepinephrine reuptake inhibitors or a pharmaceutically effective salt thereof; and
(b) a pharmaceutically effective amount of one or more antimuscarinic agents or a pharmaceutically effective salt thereof.

8. The method according to claim 7 wherein said disease or disorder is selected from the group consisting of obesity, depression, schizophrenia, a stress related disease (e.g. general anxiety disorder), panic disorder, a phobia, obsessive compulsive disorder, post-traumatic-stress syndrome, immune system depression, incontinence, a stress induced problem with the urinary, gastrointestinal or cardiovascular system, neurodegenerative disorders, autism, chemotherapy-induced vomiting, hypertension, migraine headaches, cluster headaches, sexual dysfunction in a mammal, addictive disorder and withdrawal syndrome, an adjustment disorder, an age-associated learning and mental disorder, anorexia nervosa, apathy, an attention-deficit disorder due to general medical conditions, attention-deficit hyperactivity disorder, bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, a movement disorder, oppositional defiant disorder, a pain disorder, peripheral neuropathy, post-traumatic stress disorder, premenstrual dysphoric disorder, a psychotic disorder, seasonal affective disorder, a sleep disorder, a specific developmental disorder, and selective serotonin reuptake inhibition (SSRI) “poop out” syndrome.

9. The method of claim 7 wherein said composition is administered rectally, topically, orally, sublingually, intranasally, transdermally or parenterally.

10. The method of claim 7 wherein component (a) and component (b) of said composition are simultaneously administered.

11. The method of claim 7 wherein component (a) and component (b) of said composition are concomitantly administered.

12. The method according to claim 7 wherein said disease or disorder comprises incontinence.

13. The method according to claim 12 wherein said incontinence is stress incontinence, genuine stress incontinence or mixed incontinence.

14. The method according to claim 13 wherein component (a) of said composition comprises reboxetine in its racemic or enantiomeric form and component (b) of said composition comprises tolterodine, its active metabolites or mixtures thereof.

15. The method according to claim 12 wherein said composition contains between about 0.1 mg to about 10 mg reboxetine and between about 0.05 mg to about 4 mg of tolterodine per kilogram of patient body weight.

16. A composition consisting essentially of:
(a) a pharmaceutically effective amount of reboxetine in its racemic or enantiomeric form or a pharmaceutically effective salt thereof; and
(b) a pharmaceutically effective amount of tolterodine, its active metabolites or combinations thereof or a pharmaceutically effective salt thereof;
wherein components (a) and (b) are maintained in the same or in different delivery vehicles.

17. The use of a composition comprising:
(a) a pharmaceutically effective amount of one or more norepinephrine reuptake inhibitors or a pharmaceutically effective salt thereof; and
(b) a pharmaceutically effective amount of one or more antimuscarnic agents or a pharmaceutically effective salt thereof to prepare a medicament for treating or preventing incontinence or diseases or disorders of the central nervous system.

18. The use according to claim 17 wherein component (a) comprises reboxetine in its racemic or enantiomeric form and component (b) comprises tolterodine, its active metabolites or mixtures thereof.

19. A composition comprising:
(a) a pharmaceutically effective amount of one or more norepinephrine reuptake inhibitors or a pharmaceutically effective salt thereof; and
(b) a pharmaceutically effective amount of one or more antimuscarinic agents or a pharmaceutically effective salt thereof for use as a medicament.

20. A composition comprising:
(a) a pharmaceutically effective amount of one or more norepinephrine reuptake inhibitors selected from the group consisting of tandamine, pirandamine, ciclazindol, fluparoxan, lortalamine, talsupram, talopram, prindamine, nomifensine, viloxazine, tomoxetine, duloxetine, venlafaxine, milnacipran and reboxetine and mixtures thereof or a pharmaceutically effective salt thereof; and
(b) a pharmaceutically effective amount of one or more antimuscarinic agents selected from the group consisting of tolterodine, propiverine, oxybutynin, trospium, darifenacin, temiverine and ipratropium and mixtures thereof or a pharmaceutically effective salt thereof;

21. The composition according to claim 20 wherein component (a) is reboxetine in either its racemic or +(S,S) enantiomeric form and component (b) is darifenacin.

22. The composition according to claim 20 wherein component (a) is reboxetine in either its racemic or +(S,S) enantiomeric form and component (b) is trospium.

23. The composition according to claim 20 wherein component (a) is reboxetine in either its racemic or +(S,S) enantiomeric form and component (b) is ipratropium.

24. The composition according to claim 20 wherein component (a) is duloxetine.

25. The composition according to claim 23 wherein component (b) is selected from the group consisting of tolterodine, darifenacin, trospium, ipratropium and mixtures thereof.

26. The composition according to claim 25 containing between about 1 mg to about 200 mg duloxetine and between about 0.05 mg to about 5 mg of component (b) per kilogram of patient body weight.

27. The composition according to claim 20 wherein component (a) and component (b) are maintained in the same delivery vehicle.

28. The composition according to claim 20 wherein component (a) and component (b) are maintained in different delivery vehicles.

29. A method for treating incontinence or a disease or disorder of the central nervous system in a mammal comprising administering to said mammal a pharmaceutically effective amount of a composition comprising:
(a) a pharmaceutically effective amount of one or more norepinephrine reuptake inhibitors tandamine, pirandamine, ciclazindol, fluparoxan, lortalamine, talsupram, talopram, prindamine, nomifensine, viloxazine, tomoxetine, duloxetine, venlafaxine, milnacipran and reboxetine and mixtures thereof or a pharmaceutically effective salt thereof; and
(b) a pharmaceutically effective amount of one or more antimuscarinic agents selected from the group consisting of tolterodine, propiverine, oxybutynin, trospium, darifenacin, temiverine and ipratropium and mixtures thereof or a pharmaceutically effective salt thereof.

30. The method according to claim 29 wherein said disease or disorder is selected from the group consisting of obesity, depression, schizophrenia, a stress related disease (e.g. general anxiety disorder), panic disorder, a phobia, obsessive compulsive disorder, post-traumatic-stress syndrome, immune system depression, incontinence, a stress induced problem with the urinary, gastrointestinal or cardiovascular system, neurodegenerative disorders, autism, chemotherapy-induced vomiting, hypertension, migraine headaches, cluster headaches, sexual dysfunction in a mammal, addictive disorder and withdrawal syndrome, an adjustment disorder, an age-associated learning and mental disorder, anorexia nervosa, apathy, an attention-deficit disorder due to general medical conditions, attention-deficit hyperactivity disorder, bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, a movement disorder, oppositional defiant disorder, a pain disorder, peripheral neuropathy, post-traumatic stress disorder, premenstrual dysphoric disorder, a psychotic disorder, seasonal affective disorder, a sleep disorder, a specific developmental disorder, and selective serotonin reuptake inhibition (SSRI) “poop out” syndrome.

31. The method of claim 30 wherein component (a) and component (b) of said composition are simultaneously administered.

32. The method of claim 30 wherein component (a) and component (b) of said composition are concomitantly administered.

33. The method according to claim 30 wherein said disease or disorder comprises incontinence.

34. The method according to claim 33 wherein said incontinence is stress incontinence, genuine stress incontinence or mixed incontinence.
